(19) United States
(12) Patent Application Publication
(10) Pub. No.: US 2007/0225655 A1
(43) Pub. Date: Sep. 27, 2007

(54) INTRAVAGINAL MEDICAMENT APPLICATORS

(76) Inventor: Sjoerd Osinga, Ellisville, MO (US)

Correspondence Address: Anthony G. Fussner, Suite 400, 7700 Bonhomme, St. Louis, MO, 63105, US

(21) Appl. No.: 11/684,009

(22) Filed: Mar. 9, 2007

Related U.S. Application Data
(60) Provisional application No. 60/784,900, filed on Mar. 22, 2006.

(51) Int. Cl. A61M 5/00

(52) U.S. Cl. 604/187

(57) ABSTRACT

An exemplary embodiment of a medicament applicator generally includes a body member having a reservoir portion for holding medicament and at least one outlet in communication with the reservoir portion for dispensing medicament from the reservoir portion. The applicator may also include a plunger assembly for urging medicament from the reservoir portion through the at least one outlet. The applicator may further include a removable cap configured for positioning over the dispensing end portion of the body member.

### CROSS-REFERENCE TO RELATED APPLICATION

This application claims the benefit of U.S. Provisional Application No. 60/784,900 filed Mar. 22, 2006. The disclosure of the above application is incorporated herein by reference.

### FIELD

The present disclosure generally relates to devices for delivering medicament to the vaginal cavity and its environs.

### BACKGROUND

The statements in this section merely provide background information related to the present disclosure and may not constitute prior art.

One of the disciplines of medicine is the treatment of the female reproductive system for the prevention, treatment, mitigation, diagnosis, and cure of diseases and the prevention of conception. Usually, this involves the delivery of medication and active agents to the vaginal cavity and its environs. Such agents and medications may be in the form of gels, foams, creams, suppositories, quick dissolving tablets, pastes, fluids, liquids, etc. Applicators are a common device used for delivering such medications to the vaginal cavity.

### SUMMARY

According to various aspects of the present disclosure, there are provided various exemplary embodiments of medicament applicators. In one exemplary embodiment, a medicament applicator generally includes a body member having a reservoir portion for holding medicament and at least one outlet in communication with the reservoir portion for dispensing medicament from the reservoir portion. The applicator may also include a plunger assembly for urging medicament from the reservoir portion through the at least one outlet. The applicator may further include a removable cap configured for positioning over the dispensing end portion of the body member.

In another exemplary embodiment, a medicament applicator generally includes a body member having a reservoir portion for holding medicament and at least one outlet in communication with the reservoir portion for dispensing medicament from the reservoir portion. The applicator may also include a plunger assembly having a first member and a second member slidingly associated with the first member. The first member may include a piston portion and at least two forks. The piston portion may be slidably disposed within the reservoir portion for urging medicament from the reservoir portion through the at least one outlet as the piston portion slides within the reservoir portion in a direction generally towards the at least one outlet. Each fork may include at least one interlocking member engagable with at least one interlocking member of the other fork. Engagement of the interlocking members may inhibit relative movement of the at least two forks away from each other. The second member of the plunger assembly may include at least one splitter member. The at least one splitter member may be configured for disengaging the engaged interlocking members of the at least two forks to thereby allow the at least two forks to move generally away from each other for engaging the second member. When the first and second members are engaged, the first member may be slidably moved along with the second member in a direction generally towards the at least one outlet.

An additional exemplary embodiment provides a medicament applicator that generally includes a body member. The body member includes a reservoir portion for holding medicament and at least one outlet in communication with the reservoir portion for dispensing medicament from the reservoir portion. The applicator may also include a plunger having a piston portion slidably disposed within the reservoir portion for urging medicament from the reservoir portion through the at least one outlet as the piston portion slides within the reservoir portion in a direction generally towards the at least one outlet. The applicator may be configured such that at least a portion of the piston portion may be slidably positioned at least partially through the at least one outlet external to the reservoir portion, thereby facilitating expulsion of substantially all medicament from the reservoir portion.

A further exemplary embodiment provides a medicament applicator that generally includes a body member. The body member includes a reservoir portion for holding medicament and at least one outlet in communication with the reservoir portion for dispensing medicament from the reservoir portion. The body member may also include an outer sidewall portion defining at least a portion of the exterior surface of the body member. The body member may further include an internal sidewall portion defining at least a portion of the reservoir portion. The internal sidewall portion may be spaced apart from and be generally parallel with the outer sidewall portion. The applicator may also include a plunger having a piston portion slidably disposed within the reservoir portion for urging medicament from the reservoir portion through the at least one outlet as the piston portion slides within the reservoir portion in a direction generally towards the at least one outlet. The applicator may further include a removable cap configured for positioning over the dispensing end portion of the body member. The removable cap may include a sealing portion configured to be positioned at least partially through the at least one outlet for substantially sealing the at least one outlet when the removable cap is positioned over the dispensing end portion of the body member.

Further aspects and features of the present disclosure will become apparent from the detailed description provided hereinafter. In addition, any one or more aspects of the present disclosure may be implemented individually or in any combination with any one or more of the other aspects of the present disclosure. It should be understood that the detailed description and specific examples, while indicating exemplary embodiments of the present disclosure, are intended for purposes of illustration only and are not intended to limit the scope of the present disclosure.

### DRAWINGS

The drawings described herein are for illustration purposes only and are not intended to limit the scope of the present disclosure in any way.

FIG. 1 is an exploded perspective view of a medicament applicator according to one embodiment of the present disclosure and showing an end cap, a cylindrical body portion, first and second plunger members (together forming a plunger assembly), and a grasping member disposed at an end portion of the second plunger member;

FIG. 2 is a perspective view of the medicament applicator shown in FIG. 1 with the components assembled in a compact, ready-for-transport configuration;

FIG. 3 is a cross-sectional view of the medicament applicator shown in FIG. 2;

FIG. 4 is a perspective of the medicament applicator shown in FIG. 2 with the end cap removed and the second plunger member fully drawn back and engaged with the first plunger member, thereby activating the first plunger member such that the medicament applicator is shown in a ready-to-dispense configuration;

FIG. 5 is a cross-sectional view of the medicament applicator shown in FIG. 4;

FIG. 6 is a perspective of the medicament applicator shown in FIG. 4 with the grasping member and the engaged plunger assembly shown fully depressed, thereby illustrating the medicament applicator after use;

FIG. 7 is a cross-sectional view of the medicament applicator shown in FIG. 6;

FIG. 8 is a perspective view of the body of the medicament applicator shown in FIG. 1;

FIG. 9 is a cross-sectional view of the body shown in FIG. 8;

FIG. 10 is an end elevation view of the body shown in FIG. 8 and showing the discharge outlet;

FIG. 11 is a perspective view of the first plunger member of the medicament applicator shown in FIG. 1;

FIG. 12a is an elevation view of the first plunger member shown in FIG. 11;

FIG. 12b is another elevation view of the first plunger member shown in FIG. 12a but now showing the forks with the catches engaged for holding the forks together;

FIG. 13 is a cross-sectional view of the first plunger member taken along the line 13-13 in FIG. 12a;

FIG. 14 is a cross-sectional view of the first plunger member taken along the line 14-14 in FIG. 13;

FIG. 15 is a cross-sectional view of the first plunger member taken along the line 15-15 in FIG. 14;

FIG. 16 is a cross-sectional view of the first plunger member taken along the line 16-16 in FIG. 15;

FIG. 17 is a perspective view of the second plunger member and grasping member of the medicament applicator shown in FIG. 1;

FIG. 18 is an elevation view of the second plunger member and grasping member shown in FIG. 17;

FIG. 19 is another elevation view of the second plunger member and grasping member shown in FIG. 17;

FIG. 20 is a cross-sectional view of the second plunger member and grasping member shown in FIG. 19;

FIG. 21 is a cross-sectional view of the second plunger member and grasping member taken along the line 21-21 in FIG. 19;

FIG. 22 is an end elevation view of the second plunger member and grasping member shown in FIG. 19;

FIG. 23 is a perspective view of the end cap of the medicament applicator shown in FIG. 1;

FIG. 24 is a cross-sectional view of the end cap shown in FIG. 23;

FIG. 25 is an end elevation view of the end cap shown in FIG. 23;

FIG. 26 is a cross-sectional view of the medicament applicator shown in FIG. 2 with the end cap removed, and with the grasping member and the second plunger member in a position partially drawn back from the first plunger member and the cylindrical body portion, thereby illustrating an exemplary operation of the medicament applicator;

FIG. 27 is a cross-sectional view of the medicament applicator shown in FIG. 2 with the end cap removed, and with the engaged plunger assembly partially depressed, thereby illustrating an exemplary operation of the medicament applicator; and

FIG. 28 is a perspective view of the medicament applicator shown in FIG. 2 being properly held in the hand of a user with the grasping member and second plunger member being partially drawn back.

### DETAILED DESCRIPTION

The following description is merely exemplary in nature and is in no way intended to limit the present disclosure, application, or uses. It should be understood that throughout the drawings, corresponding reference numerals indicate like or corresponding parts and features.

According to various aspects of the present disclosure, there are provided various exemplary embodiments of medicament applicators. In one exemplary embodiment, a medicament applicator generally includes a body member having a reservoir portion for holding medicament and at least one outlet in communication with the reservoir portion for dispensing medicament from the reservoir portion. The applicator may also include a plunger assembly for urging medicament from the reservoir portion through the at least one outlet. The applicator may further include a removable cap configured for positioning over the dispensing end portion of the body member.

In another exemplary embodiment, a medicament applicator generally includes a body member having a reservoir portion for holding medicament and at least one outlet in communication with the reservoir portion for dispensing medicament from the reservoir portion. The applicator may also include a plunger assembly having a first member and a second member slidingly associated with the first member. The first member may include a piston portion and at least two forks. The piston portion may be slidably disposed within the reservoir portion for urging medicament from the reservoir portion through the at least one outlet as the piston portion slides within the reservoir portion in a direction generally towards the at least one outlet. Each fork may include at least one interlocking member engagable with at least one interlocking member of the other fork. Engagement of the interlocking members may inhibit relative movement of the at least two forks away from each other. The second member of the plunger assembly may include at least one splitter member. The at least one splitter member may be configured for disengaging the engaged interlocking members of the at least two forks to thereby allow the at least two forks to move generally away from each other for engaging the second member. When the first and second members are engaged, the first member may be slidably moved along with the second member in a direction generally towards the at least one outlet.

An additional exemplary embodiment provides a medicament applicator that generally includes a body member. The body member includes a reservoir portion for holding medicament and at least one outlet in communication with the reservoir portion for dispensing medicament from the reservoir portion. The applicator may also include a plunger having a piston portion slidably disposed within the reservoir portion for urging medicament from the reservoir portion through the at least one outlet as the piston portion slides within the reservoir portion in a direction generally towards the at least one outlet. The applicator may be configured such that at least a portion of the piston portion may be slidably positioned at least partially through the at least one outlet external to the reservoir portion, thereby facilitating expulsion of substantially all medicament from the reservoir portion.

A further exemplary embodiment provides a medicament applicator that generally includes a body member. The body member includes a reservoir portion for holding medicament and at least one outlet in communication with the reservoir portion for dispensing medicament from the reservoir portion. The body member may also include an outer sidewall portion defining at least a portion of the exterior surface of the body member. The body member may further include an internal sidewall portion defining at least a portion of the reservoir portion. The internal sidewall portion may be spaced apart from and be generally parallel with the outer sidewall portion. The applicator may also include a plunger having a piston portion slidably disposed within the reservoir portion for urging medicament from the reservoir portion through the at least one outlet as the piston portion slides within the reservoir portion in a direction generally towards the at least one outlet. The applicator may further include a removable cap configured for positioning over the dispensing end portion of the body member. The removable cap may include a sealing portion configured to be positioned at least partially through the at least one outlet for substantially sealing the at least one outlet when the removable cap is positioned over the dispensing end portion of the body member.

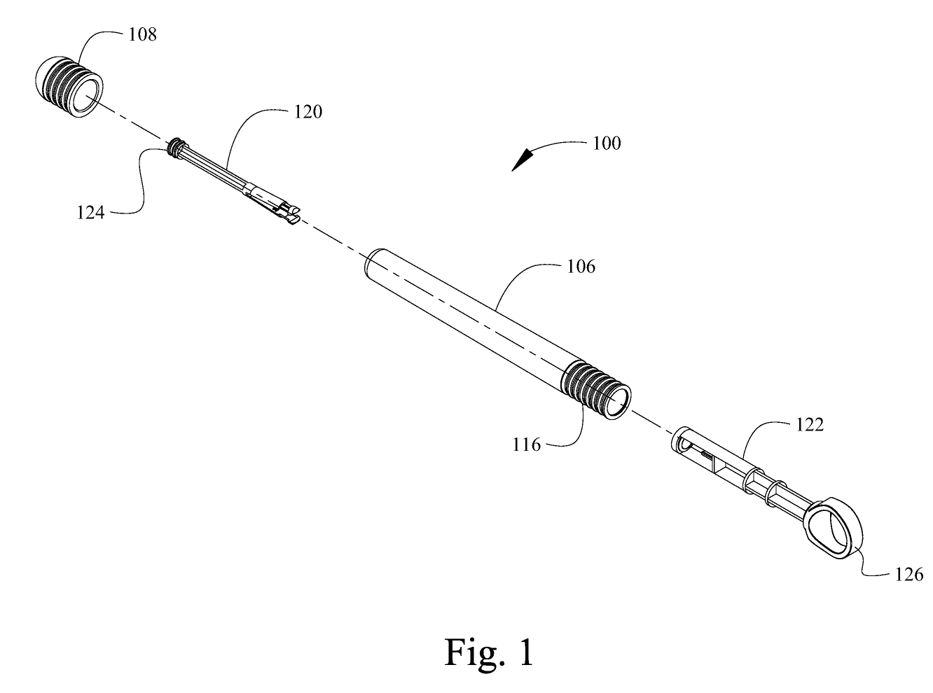

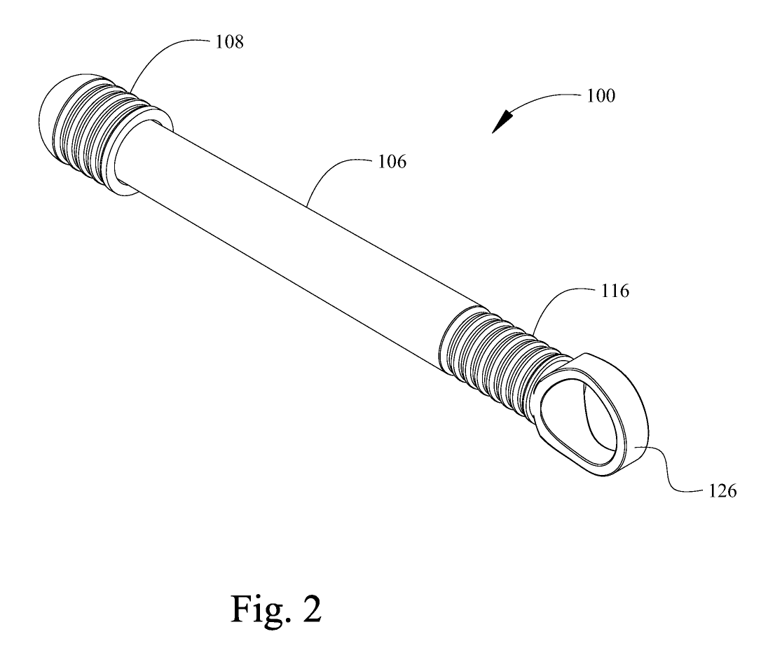

FIGS. 1 through 7 illustrate an exemplary embodiment of a medicament applicator 100 embodying one or more aspects of the present disclosure. As shown, the medicament applicator 100 includes a proximal dispensing end portion and a distal grasping end portion.

Components of the applicator 100 generally include a body member or barrel 106, a removable end cap or closure member 108, and a plunger or expulsion assembly, all of which are described in more detail herein. Briefly, the body member 106 includes a medicament reservoir portion 112 and a grasping surface portion 116. The end cap 108 is configured to be slidingly received over the dispensing end portion for substantially sealing the dispensing outlet 118 of the body member 106. The plunger assembly includes a first plunger member 120 and a second plunger member 122 slidingly associated with the first plunger member 120. The first plunger member 120 includes a piston portion 124 slidingly disposed within the reservoir portion 112 for urging medicament from the reservoir portion 112 through the outlet 118 as the piston portion 124 slides within the reservoir portion 112 in a direction generally towards the outlet 118. A grasping member 126 is disposed at a distal end portion of the second plunger member 122.

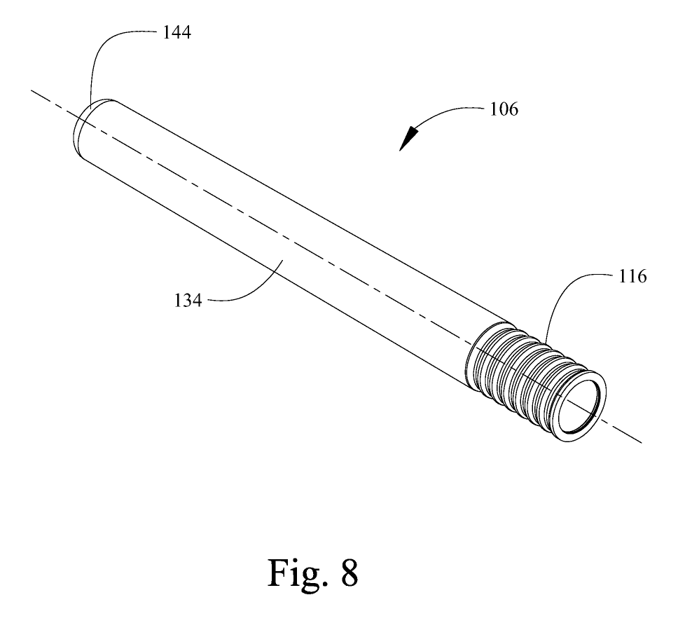

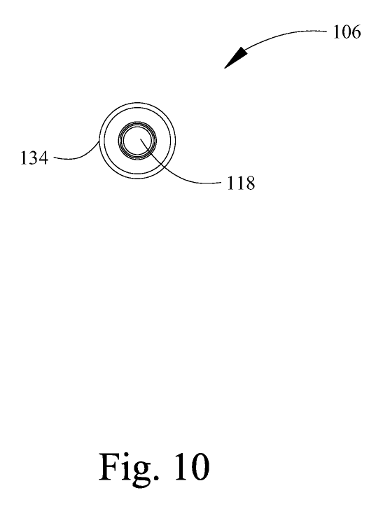

The body member 106 will now be described in more detail. Referring to FIGS. 8 through 10, one exemplary embodiment of the body member 106 is shown as a cylindrical hollow tube or barrel. In alternative embodiments, however, the body member need not be cylindrical, but other shapes would entail corresponding modification to other components of the medicament applicator.

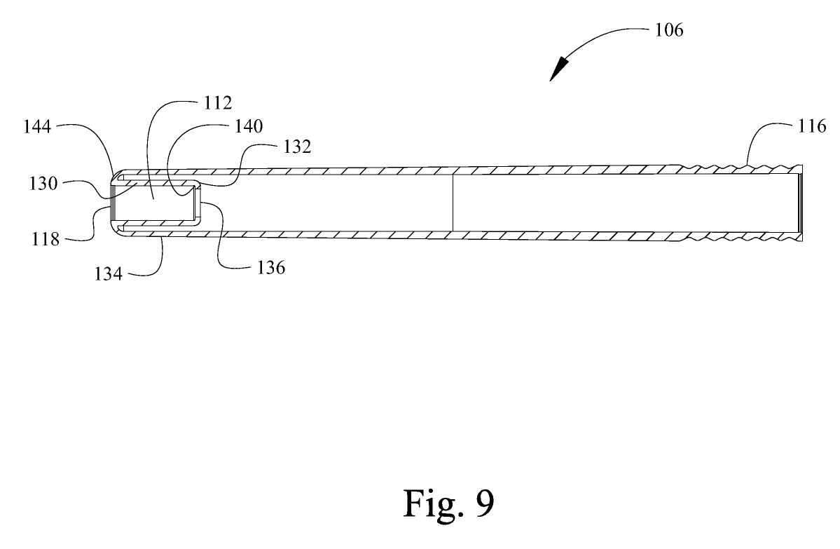

As shown in FIG. 9, the body member 106 includes internal walls 130 and 132 cooperatively defining the medicament reservoir portion 112. The medicament reservoir portion 112 is defined by the internal sidewall 130 and rearward wall 132 such that the medicament reservoir portion 112 is cylindrical. In alternative embodiments, however, the medicament reservoir portion need not be cylindrical, but other shapes would entail corresponding modification to other components of the medicament applicator.

The internal sidewall 130 is spaced apart from, generally parallel, and concentric with the outer sidewall 134 defining the cylindrical exterior surface of the body member 106. The medicament reservoir portion 112 is defined by the internal walls 130 and 132 to have a smooth inner surface and substantially constant inner diameter.

The body member 106 also includes the outlet 118. The outlet 118 is in communication with the reservoir portion 112 to allow medicament (or other materials) to be dispensed or discharged from the reservoir portion 112. As used herein, the term “outlet” broadly includes any opening (e.g., holes, perforations, nozzles, etc.) for allowing medicament to exit the reservoir portion 112. In the illustrated embodiment, only one generally circular outlet 118 is shown. Alternative embodiments, however, may include one or more circular or non-circular outlets.

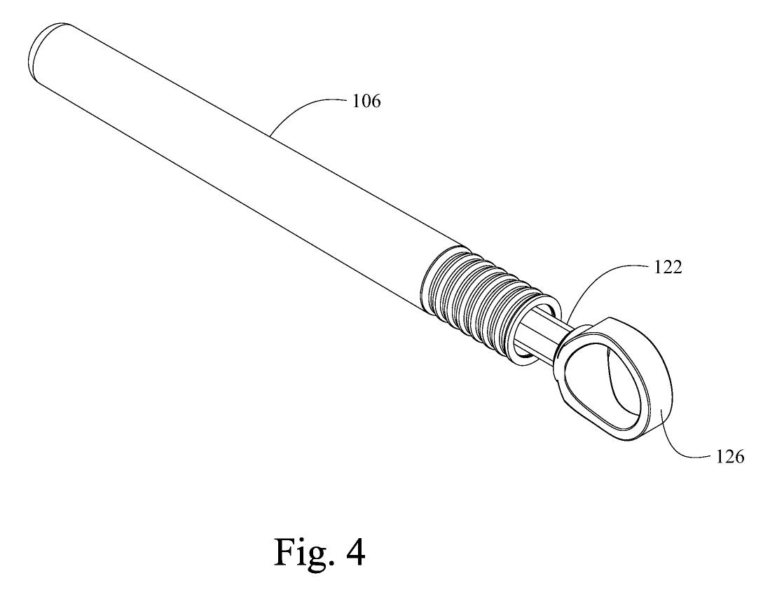

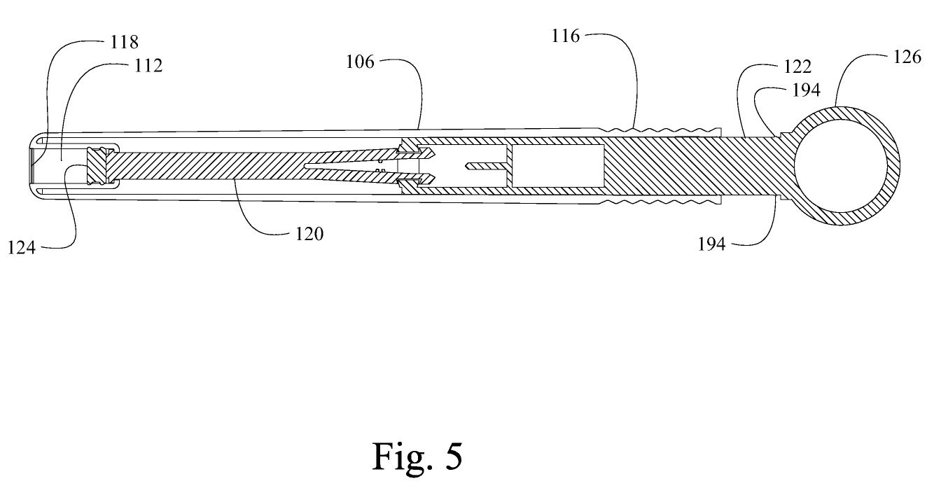

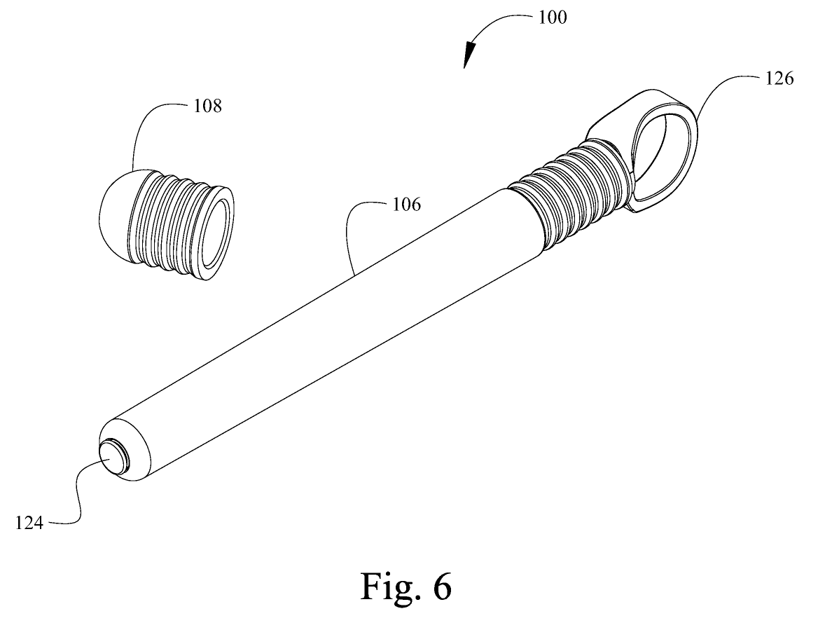

The rearward wall 132 defines an opening 136 that slidably receives therethrough at least a portion of the connecting section 138 of the first plunger member 120 (as can be seen by FIGS. 5 and 6). As shown in FIG. 5, the rearward wall 132 also operates as a stop 140 such that contact or abutment between the stop 140 and a protruding portion 142 of the first plunger member 120 inhibits the piston portion 124 from being drawn back out of the reservoir portion 112 through the opening 136, for example, when the plunger assembly is fully drawn back for use as shown in FIGS. 4 and 5. Accordingly, the stop 140 reduces the possibility of pulling the plunger's piston portion 124 out of the reservoir portion 112.

In the illustrated embodiment, the protruding portion 142 comprises a shoulder spaced apart distally from the piston portion 124. In other embodiments, however, the protruding portion 142 may comprise a distal portion of the piston portion 124 that contacts the stop 140. In still further embodiments, the stop 140 may be provided by other means. For example, the stop 140 may include a separate component affixed within the reservoir portion 112 by way of adhesives, etc.

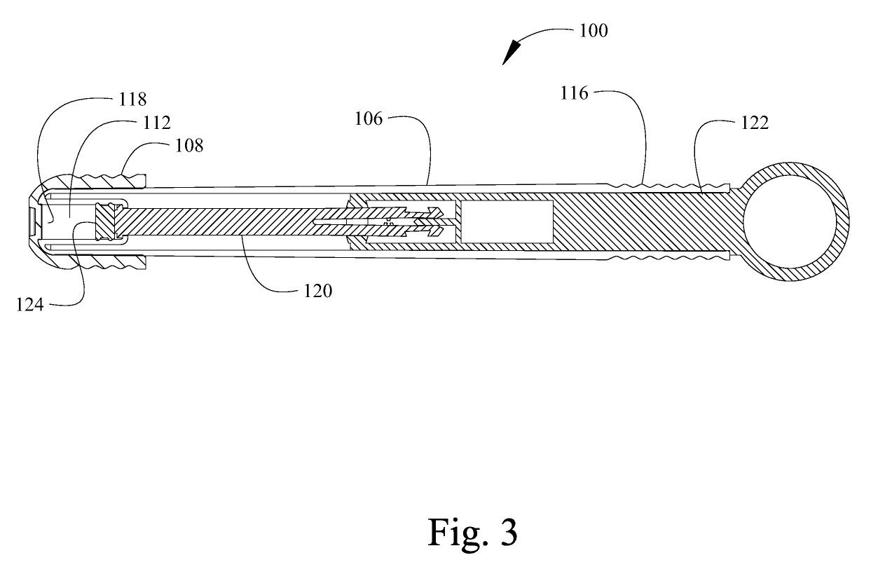

As shown in FIG. 3, placement of the piston portion 124 within the reservoir portion 112 can thus be relatively precisely controlled by way of the stop 140, which, in turn, can allow for relatively precise control of the available length, and thus volume of the reservoir portion 112.

In various embodiments, the body member 106 can also include one or more features to facilitate the handling of the medicament applicator 100. For example, the body member 106 can include a tapered end portion 144 (FIG. 8) to facilitate insertion of the applicator 100 into a body cavity. Or, for example, at least a portion of the body member 106 may be provided with one or more striations, grooves, ridges, ribs, ripples, roughened portions, etc. These exemplary features may help the user more firmly grasp the body member 106 while using the applicator 100 during insertion into a body cavity, expulsion of medicament from the reservoir portion 112 out the discharge outlet 118, and/or withdrawal of the medicament applicator 100 from the body cavity.

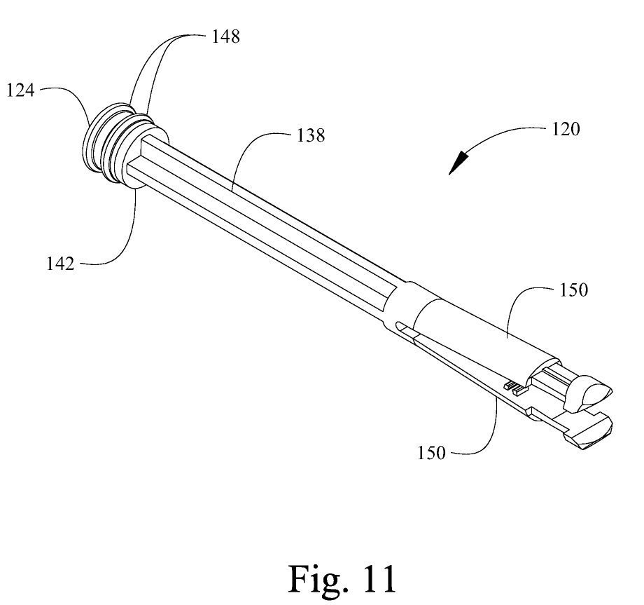

In the illustrated embodiment of FIG. 11, the body member 106 includes the outer surface portion 116 having grooves and ridges. In alternative embodiments, the body member need not be provided with any ripples, ridges, grooves, etc., but may instead include a substantially smooth exterior.

A wide range of materials may be used for the body member 106, depending, for example, on the intended use for the medicament applicator 100 and particular medication to be administered thereby. Various embodiments include a body member 106 formed from materials meeting Federal Drug Administration (FDA) requirements for use as an intravaginal applicator. By way of example only, the body member 106 in some exemplary embodiments is molded from polypropylene (e.g., Pro-fax 6323D polypropylene, etc.).

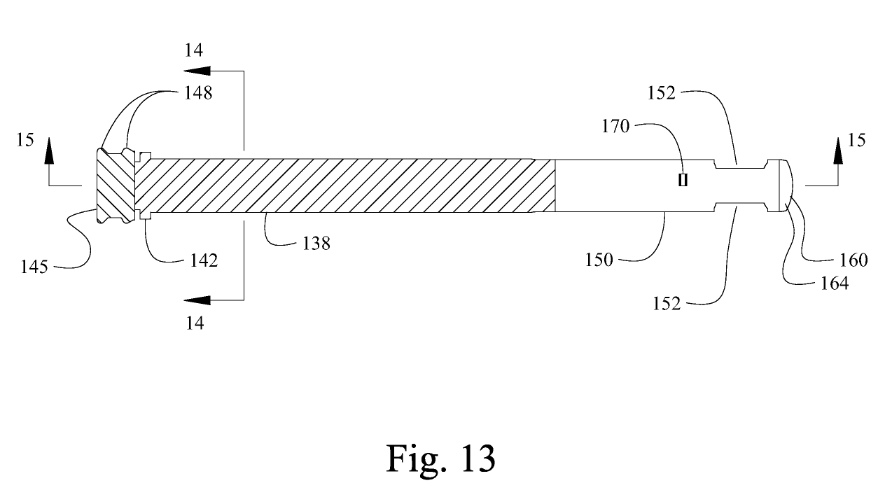

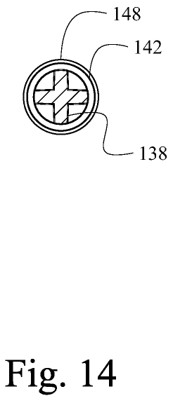

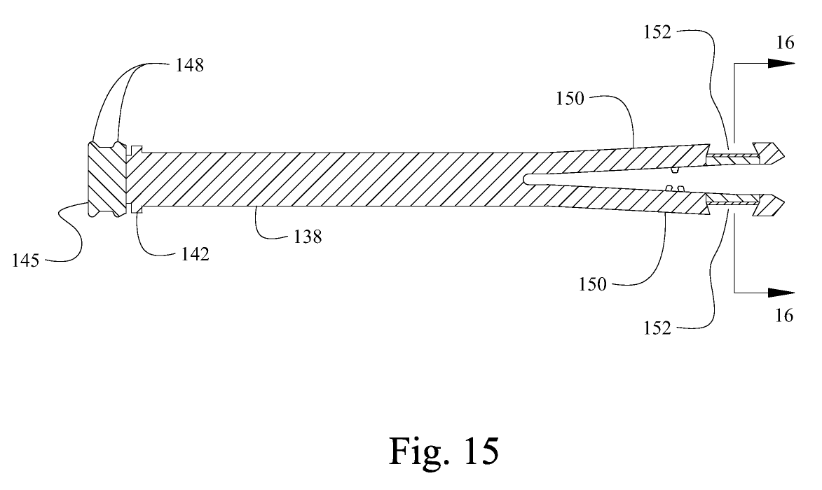

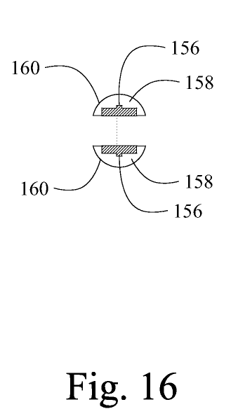

Regarding the plunger assembly, FIGS. 11 through 16 illustrate an exemplary embodiment of the first plunger member 120. As shown, the piston portion 124 of the first plunger member 120 is configured to fit within the reservoir portion 112. The piston portion 124 has a generally flattened end portion 145 for urging the medicament from the reservoir portion 112 as the piston portion 124 slides through the reservoir portion 112 (as shown by FIGS. 5, 27, and 6).

With continued reference to FIGS. 11 through 16, the piston portion 124 is configured to substantially seal the interface between the sliding surface(s) of piston portion 124 and the body member's internal sidewall 130. The sliding surfaces include those surface(s) of the piston portion 124 and the sidewall 130 that slidably contact one another as the piston portion 124 slides through the reservoir portion 112.

In the illustrated embodiment, the piston portion 124 includes two rings or annular raised rounded ridges 148 circumferentially disposed around the piston portion 124. Alternatively, the piston portion 124 may include more or less than two annular ridges depending, for example, on the particular application. Still further embodiments may include other means for sealing the interface between the sliding surfaces of the piston portion and the reservoir sidewall.

The first plunger member 120 includes two forks or forked sections 150 and a connecting section 138. The connecting section 138 is disposed generally between the piston portion 124 and the forked sections 150. The connecting section 138 includes the protruding portion 142, which in this illustrated embodiment is a shoulder. As shown in FIG. 5, the protruding portion 142 can contact or abut against the stop 140 within the reservoir portion 112 to inhibit the piston portion 124 from being drawn back out of the reservoir portion 112.

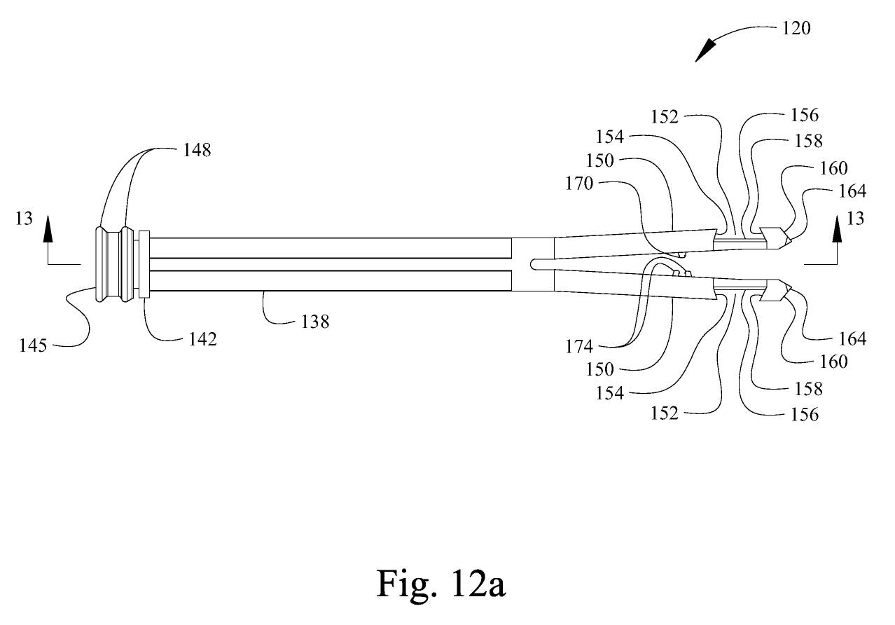

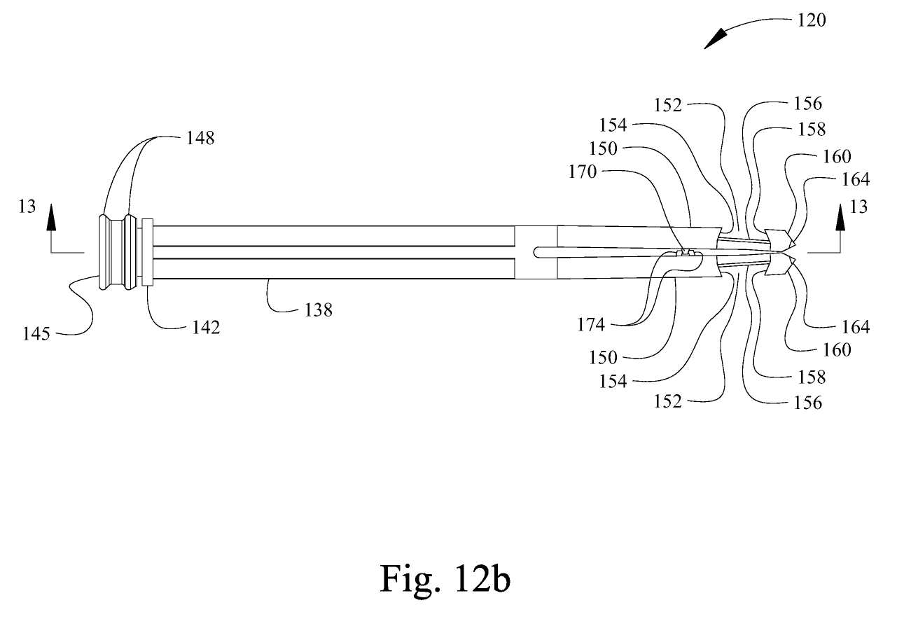

As shown in FIG. 12, each fork 150 has a recessed or notched section 152 having a sloped surface or shoulder 154, a reduced thickness midsection 156, and a sloped distal surface or shoulder 158 leading to an enlarged end portion 160. Accordingly, this particular illustrated embodiment includes two substantially identical forks 150. In other embodiments, however, the forks may have configurations different from each other.

The notched sections 152 cooperate with corresponding lip portions 162 of the second plunger member 122 to interlock or engage the first plunger member 120 to the second plunger member 122, as shown in FIG. 5 and described in more detail herein.

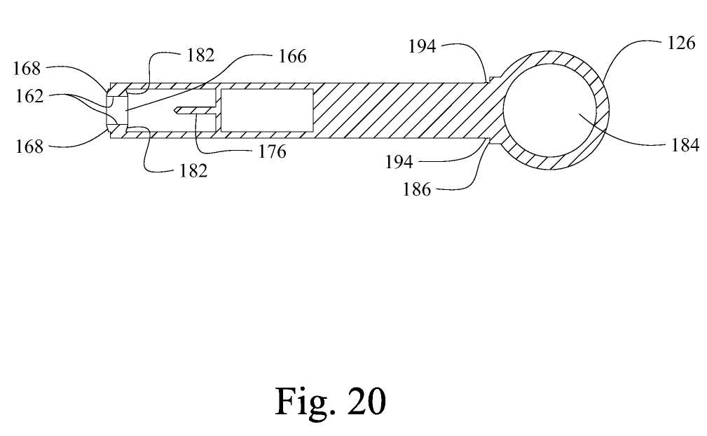

With further reference to FIG. 12, the enlarged end portions 160 of the forks 150 include sloped distal surfaces 164. These slanted distal surfaces 164 can operate as camming surfaces for urging the forks 150 towards each other, and facilitate insertion of the forks 150 through the outlet 118, through the opening 136 defined by the rearward wall 132 of the reservoir portion 112, and/or through the opening 166 defined by the second plunger member 122. For example, during an exemplary assembly process, the forks 150 may be inserted through the discharge outlet 118 and the opening 136 out of the reservoir portion 112. This insertion may position the fork's camming surfaces 164 in contact with the rearward wall 132, thereby urging the forks 150 towards each other. Or, for example, the fork's camming surfaces 164 may contact correspondingly slanted or sloped surfaces 168 of the second plunger member 122 (FIG. 20) as the forks 150 are inserted through the opening 166 of the second plunger member 122. Accordingly, the contact between the fork's distal camming surfaces 164 and the second plunger member's proximal camming surfaces 168 can urge the forks 150 towards each other, thereby facilitating insertion of the fork's 150 through the second plunger member's opening 166.

In various embodiments, snap or catch features (e.g., interlocking members, corresponding male and female catches, keys and keyways, etc.) are provided to help hold the forked sections 150 together, for example, as the first plunger member 120 is being assembled into the body member 106. By way of example only, exemplary male catch features include one or more ribs, projections, protrusions, ridges, detents, dimples, ramp-ups, etc. Exemplary female catch features include one or more corresponding or reciprocally-configured openings, recesses, voids, cavities, slots, grooves, holes, depressions, etc. The engagement of the male catch feature(s) with the female catch feature(s) can help hold the forked sections together.

In the particular illustrated embodiment of FIG. 12A, one of the forks 150 includes a first projection or protrusion 170, and the other fork 150 includes an opening defined generally between two other projections or protrusions 174. The opening is configured to engagingly receive the projection 170 for inhibiting relative movement of the forks 150 away from each other, as shown in FIG. 12B. Alternative embodiments, however, can include different male and female catch features than that shown in the figures and described herein. Still further embodiments do not include any of such male and female catch features.

As shown in FIG. 3, the second plunger member 122 includes one or more splitters 176 (e.g., ribs, spikes, etc.) for decoupling or disengaging the male and female catches 170, 174 of the first plunger member 120. The splitter 176 can also be configured (e.g., sized, shaped, located, etc.) for helping to maintain the forks 150 spread apart from one another, for example, during storage. In some embodiments, the splitter 176 can also be configured to induce “cold forming” of the forks 150 to thereby help maintain elasticity of the forks 150, for example, during storage of the applicator 100, which, in turn, helps ensure a workable applicator even after the passage of time.

In some embodiments, the spread-apart forks 150 are configured to frictionally engage and form an interference/friction fit with sidewall portions of the second plunger member 122. For example, and with continued reference to FIG. 3, the forks 150 may frictionally engage sidewall portions defining the second plunger member's opening 166. Additionally, or alternatively, outer surfaces of the forks 150 may frictionally engage other sidewall portions of the second plunger member 122. The frictional engagement between the first and second plunger members 120 and 122 can help maintain their relative positioning. This, in turn, tends to help avoid premature activation of the plunger assembly and to inhibit the piston portion 124 from accidentally sliding towards the dispensing outlet 118 and causing premature medicament discharge.

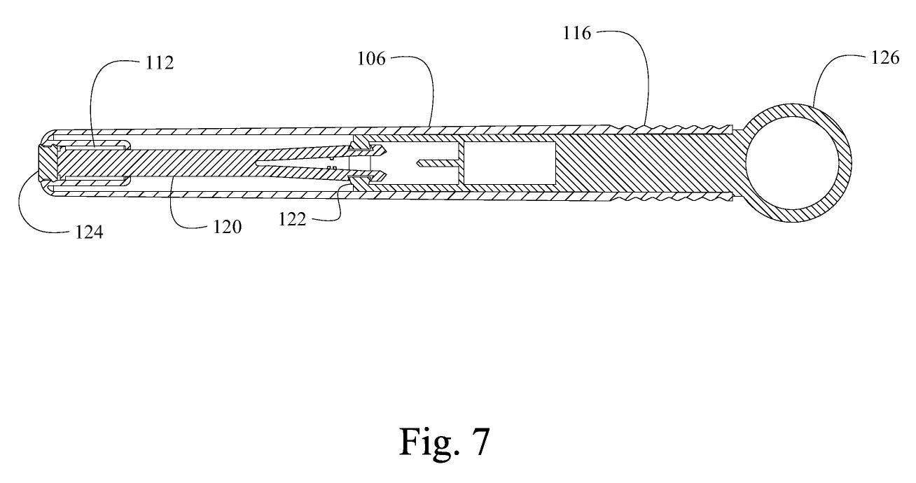

As shown in FIGS. 6 and 7, various components of the applicator 100 are configured such that the piston portion 124 extends at least partially out through the outlet 118, thereby helping ensure complete expulsion of all (or at least substantially all) medicament from the reservoir portion 112. In one particular embodiment, a portion of the piston portion 124 extends about one-eighth inch externally beyond the outlet 118 when the plunger assembly has been fully depressed as shown in FIGS. 5 and 6. Alternative embodiments, however, are configured such that the piston portion extends more or less than one-eighth inch out of the outlet depending, for example, on the intended use for the medicament applicator 100 and particular medicament (e.g., viscosity, etc.) to be administered thereby. In addition, some embodiments are configured such that the piston portion remains entirely within the reservoir portion during use, such as, for example, embodiments intended to dispense relatively free-flowing medicaments that can be readily dispensed from a reservoir portion without having the piston portion extend outwardly beyond the outlet.

Various embodiments include medicament applicators configured to produce an indicator (such as an audible “click” and/or tactile “click”) for indicating to the user that the medicament has been expelled from the applicator. By way of example, the second plunger member 122 may include one or more protruding portions 194 adjacent the grasping member 126. The one or more protruding portions 194 can be configured to produce an audible and/or tactile indicator when the protruding portions 194 are received within the body member 106. The one or more protruding portions 194 can be positioned relative to the second plunger member 122 such that the one or more protruding portions 194 are received within the body member 106 (and thus produce the audible and/or tactile indicator) substantially simultaneously with the passage of the at least a portion of the piston portion 124 through the outlet 118. Accordingly, the detection of the “click” or other suitable indicator informs the user that the piston portion 124 has traveled completely through the reservoir portion 112, thus meaning that all (or substantially all) medicament has been completely expelled from the applicator 100. Given that the applicator's reservoir portion 112 will not be visible to the user during normal usage, having the applicator 100 produce such a tactile and/or audible indicator can help ensure delivery of the entire medicament dosage with a single use of the medicament applicator. Additionally, or alternatively, various embodiments include a piston portion 124 formed from one or more materials having sufficient resiliency to permit compression of the piston portion 124 for fitting at least partially through the outlet 118, and to respond with a sufficient restorative force such that an audible and/or tactile indicator is produced by way of the expansion of that portion of the piston portion 124 extending through the outlet 118.

A wide range of materials can be used for the first plunger member 120 depending, for example, on the intended use for the medicament applicator 100 and particular medicament to be administered thereby. For example, various embodiments include the first plunger member 120 being formed from materials meeting FDA requirements for use as intravaginal applicators. Preferably, the forks 150 of the first plunger member 120 are made of one or more materials resilient enough to permit compression of forks 150 towards each other and to respond with restorative force to urge the forks 150 to spread or spring apart away from each other. In addition, the piston portion 124 is also preferably formed from one or more materials having sufficient resiliency to allow the raised ridges 148 of the piston portion 124 to form a substantially fluid-tight seal with the inner surface of the reservoir portion 112 defined by the sidewall 130. In some embodiments, the one or more materials forming the piston portion 124 are preferably resilient enough to permit compression of the piston portion 124 for fitting at least partially through the outlet 118.

Moreover, the same material need not be used for each component or portion of the first plunger member 120. By way of example only, various embodiments include a shot-molded first plunger member with a piston portion formed from rubber or other resilient material (e.g., Santoprene® rubber material, etc.), and with the remainder (the connecting section and forks) formed from polypropylene (e.g., Pro-fax 6323B polypropylene, etc.). In such exemplary embodiments, the piston portion 124 can thus be resiliently compressible for fitting at least partially through the outlet 118, while the protruding portion 142 of the connecting section 138 is formed from a more rigid material(s) having sufficient rigidity for contacting the stop 140 and inhibiting the protruding portion 142 (and thus piston portion 124) from being pulled through the opening 136 of the rearward wall 132.

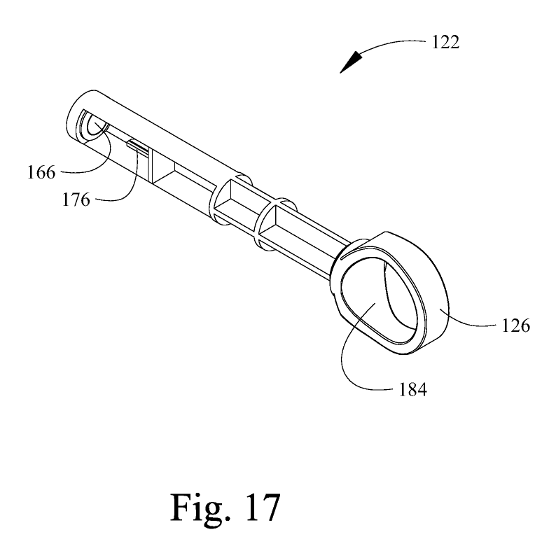

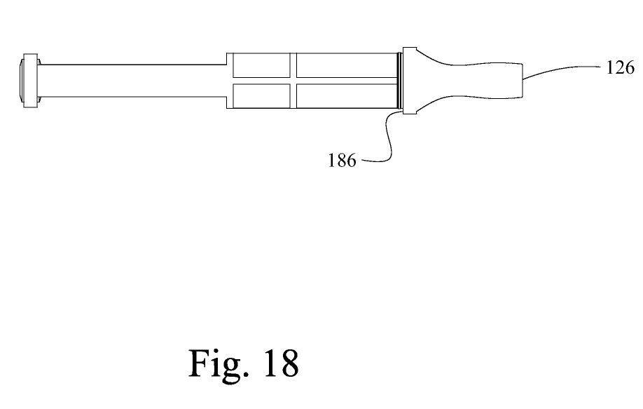

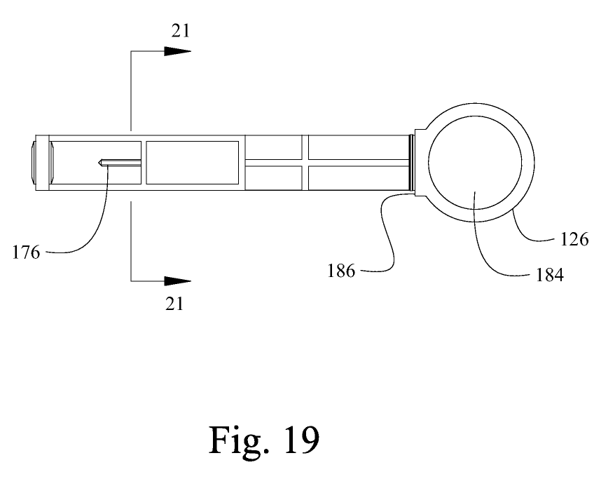

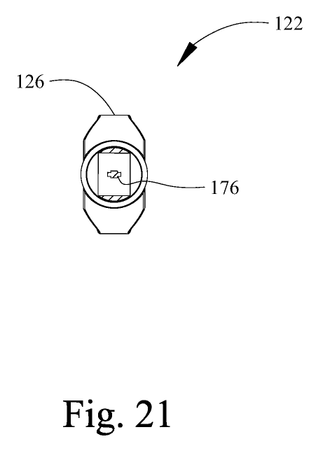

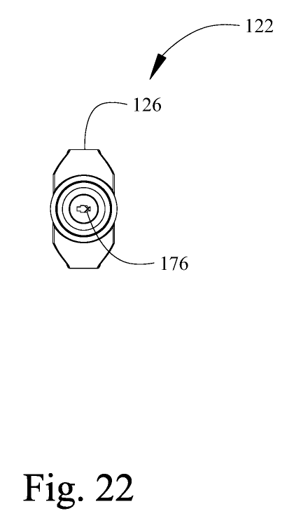

Continuing with the description of the plunger assembly, an exemplary embodiment of the second plunger member 122 is shown in FIGS. FIGS. 17 through 22. As shown, the second plunger member 122 includes the lip portions 162 and opening 166. The lip portions 162 include proximally disposed sloped surfaces 168 and distally disposed sloped surfaces 182.

The surfaces 168,182 of the second plunger member 122, and the reciprocally slanted surfaces 154, 158 of the forks 150 are oriented (as shown in FIG. 5) so as to inhibit disengagement of the first plunger member 120 from the second plunger member 122, for example, if the second plunger member 122 (or grasping member 126) is rotated as the second plunger member 122 is being drawn back to activate the plunger assembly. The surfaces 154, 168 have reciprocally slanted orientations for producing a force in a direction generally radially outward relative to a longitudinal centerline of the applicator 100, as the first plunger member 120 is slidably move along with the second plunger member 122 in a direction towards the outlet 118 (see FIGS. 5, 27, and 7). The surfaces 158, 182 have reciprocally slanted orientations so as to inhibit disengagement of the lip portions 162 from the recessed portion 152 of the forks 150 when a force is applied to the second plunger member 122 in a direction generally opposite the outlet 118, such as when the second plunger member 122 is pulled after engagement of the forks 150 and lip portions 162. This can also inhibit disengagement, for example, even when the second plunger member 122 (or grasping member 126) is rotated as the second plunger member 122 is being drawn back to activate the plunger assembly. Accordingly, the orientations of the slanted surfaces 154, 158, 168, 182 can help generate forces for creating a more positive interlock or engagement between the first and second plunger members 120 and 122, thus inhibiting their disengagement during use of the applicator 100.

In addition, the proximally disposed slanted surfaces 168 of the second plunger member 122 can operate as camming surfaces, in cooperation with the distal slanted end surfaces 164 of the forks 150, for urging the forks 150 towards each other as the applicator 100 is being assembled.

The second plunger member's slanted surfaces 168, 182 engage or interlock with the corresponding or reciprocally slanted surfaces 154, 158 of the forks 150. More specifically, the second plunger member 122 can be drawn back in a direction opposite the outlet 118 (as can be seen by FIGS. 3, 26, 5) until the lip portions 162 of the plunger member 122 are generally aligned with the recessed portions 152 of the forks 150. At that point, the forks 150 spring generally outward, thus interlocking the second plunger member's lip portions 162 within the forks' recessed portions 152. As shown in FIG. 5, the sloped surfaces 154 cooperate with the sloped surfaces 168 to inhibit forward slippage, and sloped surfaces 158 cooperate with sloped surface 182 to inhibit back slippage. In addition, the engagement of the first plunger member's surfaces 154 and 158 with the second plunger member's surfaces 168, 182, in cooperation with the abutment and contact between the first plunger member's protruding portion 142 and stop 140 within the reservoir portion 112, can operate as a “stop” indicating that the plunger assembly is fully drawn back and in a ready-to-dispense configuration.

In some embodiments, the first and second plunger members 120 and 122 are configured such that an audible and/or tactile indicator (e.g., a “click”, etc.) is produced when the first plunger member 120 is activated. By way of example only, an audible and/or tactile “click” may be produced by the forks 150 springing generally outward and interlocking the second plunger member's lip portions 162 within the recessed portions 152 of the forks 150.

With further reference to FIGS. 17 through 22, the grasping member 126 is disposed at the distal end portion of the second plunger member 122. In this illustrated embodiment, the grasping member 126 is in the shape of a ring having an opening 184 for insertion of the user's finger. Alternative configurations are also possible for the grasping member, such as non-circular annular members and other means for facilitating grasping and handling of the second plunger member.

Further, the grasping member 126 is shown as an integral part of the second plunger member 122. The grasping member 126 and second plunger member 122 have a monolithic construction. With this integral or monolithic construction, the grasping member 126 is inhibited from being separated from the second plunger member 122, for example, when the second plunger member 122 is being drawn back to activate the plunger assembly. In alternative embodiments, however, the grasping member 126 may comprise a separate component that is engaged to the second plunger member 122, for example, by an interference fit, threaded connection, adhesives, etc.

In various embodiments, the second plunger member 122 can include a stop configured to rest positively against the end of the body member 106, that is, except when the plunger assembly is being drawn back or depressed during use of the applicator 100. In the illustrated embodiment, the stop comprises an external shoulder 186 configured to contact and abut against the distal end of the body member 106, as shown in FIG. 3.

A wide range of materials can be used for the second plunger member 122 depending, for example, on the intended use for the medicament applicator 100 and particular medicament to be administered thereby. For example, various embodiments include the second plunger member 122 being formed from materials meeting FDA requirements for use as intravaginal applicators. By way of example only, the second plunger member 122 in some exemplary embodiments is molded from polypropylene (e.g., Pro-fax 6523D polypropylene, etc.).

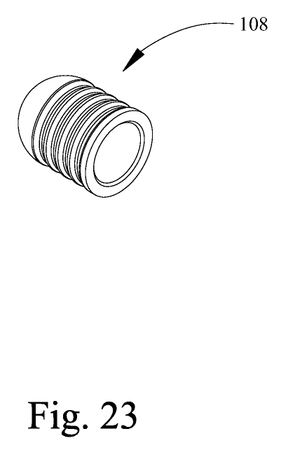

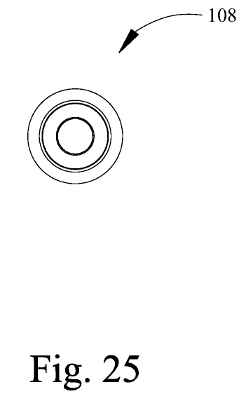

FIGS. 23 through 25 illustrate an exemplary embodiment of the end cap 108. The end cap 108 is configured to be positioned over the dispensing end portion for substantially sealing the discharge outlet 118 of the body member 106.

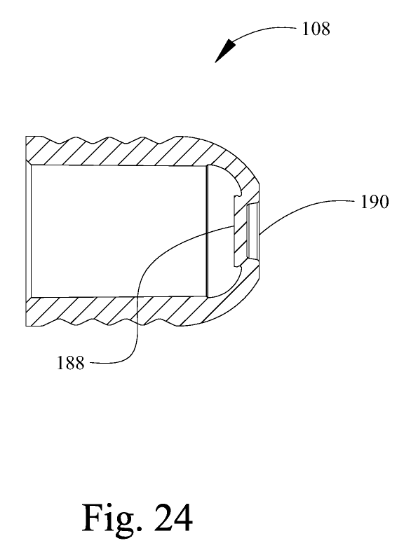

As shown in FIG. 24, the end cap 108 includes a sealing portion or member 188. This sealing member 188 is configured to be received at least partially through the discharge outlet 118 into the reservoir portion 112. Accordingly, placing the end cap 108 over the dispensing end portion includes positioning at least a portion of the sealing member 118 at least partially through the outlet 118 into the reservoir portion 112. This, in turn, allows the reservoir portion 112 to be substantially hermetically sealed by the end cap 108 in cooperation with the raised annular ridges 148 of the piston portion 124. This hermetic sealing can help keep air away from the product or medicament within the reservoir portion 112, and also help retain the product or medicament within the reservoir portion 112 over a predetermined amount of time. Accordingly, some embodiments provide a single use, pre-filled applicator 100 that can contain and store medication over a pre-established amount of time without having the medication using its efficacy (stability).

As shown in FIG. 24, the end cap 108 includes a recessed portion or pocket 190. This recessed portion 190 can provide a degree of compression to the end cap 108 and its sealing member 188, to thereby facilitate positioning of at least a portion of the sealing member 188 through the outlet 118 into the reservoir portion 112.

In various embodiments, the end cap 108 also includes one or more features to facilitate handling of the end cap 108, such as removal from the dispensing end portion. For example, the end cap's exterior or outer surface may be provided with one or more striations, grooves, ridges, ribs, ripples, roughened portions, etc. In the illustrated embodiment, the end cap 108 includes grooves 192 that make the end cap 108 easier to grip, for example, when removing the end cap 108 from the medicament applicator 100. Alternative embodiments, however, include an end cap having a substantially smooth exterior without any ribs, ripples, ridges, etc.

A wide range of materials can be used for the end cap 108, depending, for example, on the intended use for the medicament applicator 100 and particular medication to be administered thereby. Various embodiments include an end cap formed from materials meeting FDA requirements for use as an intravaginal applicator. Preferably, the end cap 108 is made of one or more materials resilient enough to permit compression of the end cap's sealing member 188 for fitting at least partially through the discharge outlet 118 into the reservoir portion, and to respond with a sufficient restorative force for expansion of the end cap's sealing member 188 for substantially sealing the outlet 118. By way of example only, the end cap 108 in some exemplary embodiments is molded from polypropylene with rubber additive (e.g., Pro-fax 6523 polypropylene with a Santoprene® rubber additive, etc.).

The applicator 100 can be specifically configured or tailored for delivering a predetermined dosage of a particular medicament. In one example, the applicator components are configured for delivering a one hundred milligram dose of Danazol synthetic androgen. In this particular example, the reservoir portion 112, first plunger member 120 (and its piston portion 124), and the end cap's sealing member 188 are all configured such that the reservoir portion 112 (with the sealing member 188 and piston portion 124 therein as shown in FIG. 3) can hold a one hundred milligram dose of Danazol, or thereabout. In another particular example, the applicator components are configured for delivering a two hundred milligram dose of Danazol synthetic androgen. The particular dosages and medicament set forth in this paragraph are mere examples and can be varied depending, for example, on the intended use for the medicament applicator 100 and particular medicament to be administered thereby.

The exploded view of FIG. 1 is illustrative of one exemplary mode of assembly for the applicator 100. In this particular example, the forks 150 of the first plunger member 120 are squeezed or pinched together such that the catches 170 and 174 are engaged and hold the forks 150 together, as shown in FIG. 12b. The first plunger member 120 (with its catch features 170 and 174 engaged) is inserted distal end first through the outlet 118, reservoir portion 112, and opening 136 of the body 106. During this insertion, the sloped distal facing surfaces 164 of the forks 150 can operate as camming surfaces for urging the forks 150 towards each other, which may facilitate insertion as the first plunger member 120 is inserted through the outlet 118, reservoir portion 112, and opening 136 of the rearward wall 132. Insertion of the first plunger member 120 continues until the first plunger member's protruding portion 142 contacts the stop 140 within the reservoir portion 112 (see the position illustrated in FIG. 3).

The second plunger member 122 is inserted into the distal end portion of the body member 106. During this insertion, the slanted surfaces 168 of the second plunger member 122 may operate as camming surfaces for urging the forks 150 towards each other, which may facilitate insertion of the forks 150 through the opening 166 of the second plunger member 122. The forks 150 are positioned through the second plunger member's opening 166. The splitter member 176 of the second plunger member 122 causes decoupling or disengagement of the forks' catches 170 and 174 (FIGS. 3 and 12b). This allows the forks 150 to spread apart from each other (FIG. 12b). Insertion of the second plunger member 122 continues until the second plunger member's stop 186 contacts the distal end of the body member 106 (see the position illustrated in FIG. 3).

The reservoir portion 112 can be filled through the outlet 118, before the second plunger member 122 is assembled. This particular sequence can depend on the medicament filling equipment and procedure that is used. After the reservoir 120 has been filled, the end cap 108 can be positioned over the dispensing end portion with the end cap's sealing member 188 extending at least partially through the outlet 118 into the reservoir portion 112. The end cap's sealing member 188 cooperates with the raised annular ridges 148 of the piston portion 124 to hermetically seal the reservoir portion 112. The applicator 100 is now ready for distribution for use as a prefilled, single-use applicator, with the hermetically sealing helping to keep air away from the product or medicament within the reservoir portion 112 and helping to retain the product or medicament within the reservoir portion 112 over a predetermined amount of time. Accordingly, some embodiments provide a single use, pre-filled applicator 100 that can contain and store medication over a pre-established amount of time without having the medication using its efficacy (stability).

The following is a description of an exemplary operation of the medicament applicator 100 provided for use with the reservoir portion 112 prefilled with a desired medicament dosage. Generally, the end cap 108 is removed from the applicator 100. The second plunger member 122 is drawn back to engage the first and second plunger members 120 and 122. The applicator 100 is then inserted into the body cavity. The plunger assembly is depressed such that the piston portion 124 slides through the reservoir portion 112 urging medicament from the reservoir portion 112 out the discharge outlet 118. The applicator 100 is then withdrawn and discarded.

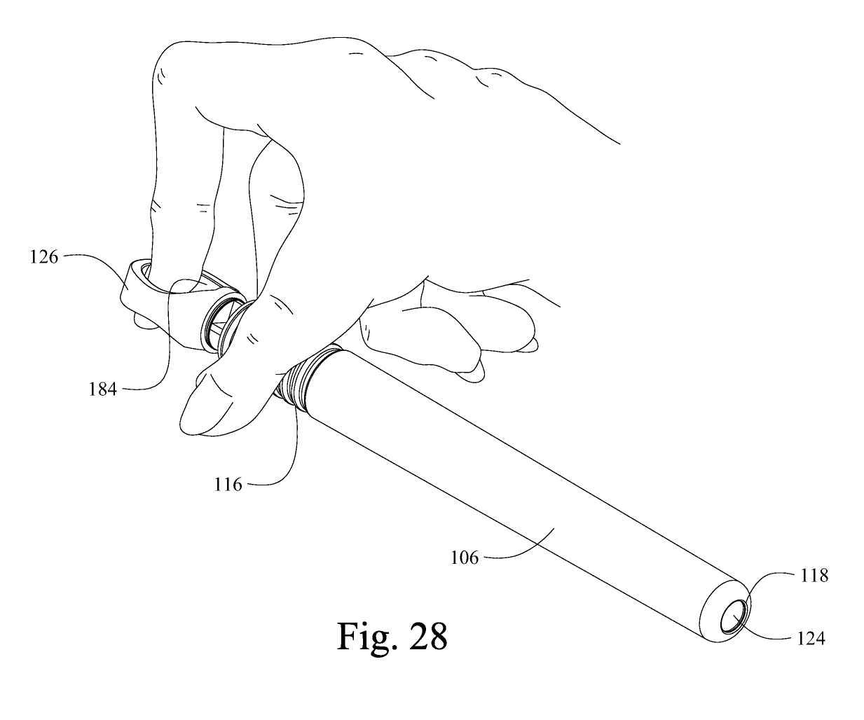

As shown in FIG. 28, the applicator 100 is intended to be hand held, for example, with the index finger through the ring opening 184, the grasping portion 116 between the thumb and at least one remaining finger, and the dispensing outlet 118 directed towards the user. For intravaginal delivery, the applicator 100 is placed, dispensing end first, into the vaginal orifice and inserted upwards approximately until the grasping portion 116 reaches the area surrounding the orifice. The rippled or ringed grasping portion 116 of the body member 106 can be configured for providing a tactile indication that the applicator 100 has been inserted to the desired depth for the “average-sized” woman. The depth of such insertion is preferably kept within the limits of comfort for the user, as there will be variations in vaginal depth.

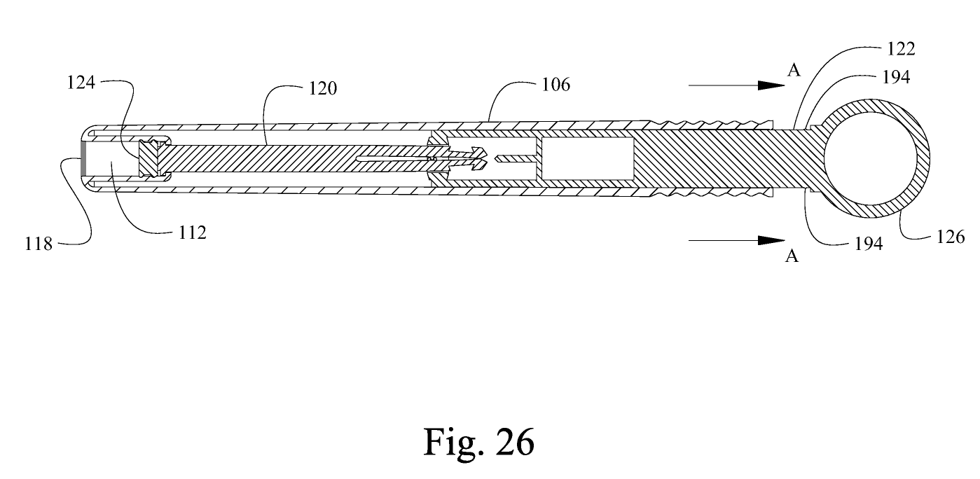

With the applicator 100 properly inserted, the user's index finger is extended to thereby cause the grasping member 126 and the second plunger member 122 to be drawn back in the direction of arrows A (FIG. 26) until the plunger members 120 and 122 are engaged with each other (FIGS. 4 and 5). This engagement includes the spread-apart or open forks 150 of the first plunger member 120 lock into position around the lip portions 162 of the second plunger member 122 with the lip portions 162 firmly seated within the fork's notched sections 152. In some embodiments, the engagement of the first plunger member 120 with the second plunger member 122 may produce an audible and/or tactile indicator (e.g., a “click”, etc.) indicating to the user that the applicator 100 is ready to dispense medicament. In addition, continued rearward movement of the plunger assembly may be inhibited by the stop 140 within the reservoir portion 112.

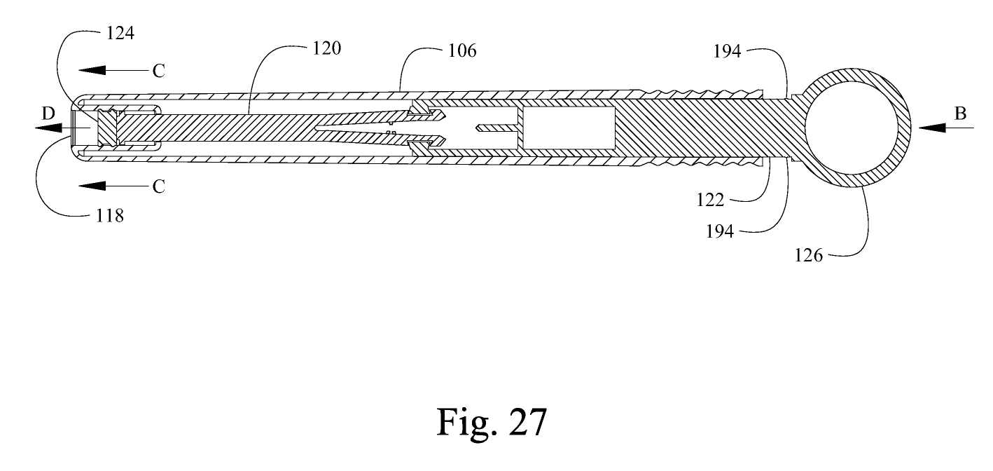

The user's index finger is flexed to drive the plunger assembly toward the discharge outlet 118 (see arrow B in FIG. 27), causing pressure to be exerted on the piston portion 124. In response, the piston portion 124 slides towards the outlet 118 (see arrows C) urging medicament from the reservoir portion 112 out the outlet 118 (see arrow D) and into the vaginal cavity. The plunger assembly should be continually pressed forward until the shoulder 186 contacts the end of the body member 106 (FIGS. 6 and 7).

In some embodiments, the piston portion 124 will extend at least partially out through the outlet 118 (as shown in FIGS. 6 and 7), which helps ensure complete expulsion of all medicament from the reservoir portion 112. In some exemplary embodiments, the applicator 100 is configured to produce an audible and/or tactile indicator that indicates to the user that the medicament has been expelled from the applicator 100. The applicator 100 may then be withdrawn and discarded.

Accordingly, aspects of the present disclosure provide various embodiments of medicament applicators that can be used for delivering medicament to the vaginal cavity (or other body cavities) and its environs. Some embodiments include applicators that are intended to be used as single use, pre-filled applicators designed to contain and store medication over a pre-established amount of time without having the medication using its efficacy (stability).

Additional aspects of the present disclosure provide body members, plunger assemblies, first plunger members, second plunger members, end caps, and combinations thereof. Further aspects of the present disclosure include applicators that include such body members, plunger assemblies, first plunger members, second plunger members, and/or end caps. Other aspects of the present disclosure include methods relating to assembling applicators, and methods relating to using applicators. Any one or more aspects of the present disclosure may be implemented individually or in any combination with any one or more of the other aspects of the present disclosure.

Various embodiments of the present disclosure provide medicament applicators that are particularly suited for delivering highly viscous materials at fairly low dosages to the vaginal cavity (and other body cavities). Medicament applicators of the present disclosure may also be employed to deliver medicaments to body cavities other than the vagina, for example, the anus, or to deliver medicaments to locations other than the inside of body cavities. Further, applicators of the present disclosure are not limited to use in human beings, and could be used for delivering medication to animals, such as delivering worming medicine to horses.

By way of example only, some embodiments include an applicator specifically configured for delivering a one hundred milligram dose of Danazol, while other embodiments include an applicator specifically configured for delivering a two hundred milligram dose of Danazol. But applicators of the present disclosure may also be used to dispense other materials besides Danazol, such as fluids, pastes, creams, low viscosity materials. Plus, aspects of the present disclosure can also be used for delivery materials having no medicinal purposes (e.g., delivering a placebo in an experimental context, etc.). Accordingly, aspects of the present disclosure should not be limited to use with any specific form/type of deliverable. Moreover, exemplary embodiments can be configured for delivering other dosage amounts besides one hundred milligram doses or two hundred milligram doses. Accordingly, aspects of the present disclosure should not be limited to applicators that can deliver only one specific dosage. Depending on the particular application, however, it may be desired to modify the particular configuration (e.g., shape, size, diameter, etc.) of one or more of the applicator components for such other uses.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order or performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A medicament applicator comprising:
a body member including a reservoir portion for holding medicament, and at least one outlet in communication with the reservoir portion for dispensing medicament from the reservoir portion;
a plunger assembly including a first member and a second member slidingly associated with the first member;
the first member including a piston portion and at least two forks, the piston portion slidably disposed within the reservoir portion for urging medicament from the reservoir portion through the at least one outlet as the piston portion slides within the reservoir portion in a direction generally towards the at least one outlet, each said fork including at least one interlocking member engagable with at least one interlocking member of the other said fork, whereby engagement of the interlocking members inhibits relative movement of the at least two forks away from each other; and
the second member including at least one splitter member configured for disengaging the engaged interlocking members of the at least two forks to thereby allow the at least two forks to move generally away from each other for engaging the second member such that the first member is slidably movable along with the second member in a direction generally towards the at least one outlet.

2. The medicament applicator of claim 1, wherein the splitter member is configured to help maintain a spaced distance between the at least two forks after disengagement of the interlocking members, whereby the splitter member helps the at least two forks maintain elasticity.

3. The medicament applicator of claim 1, wherein the applicator is configured such that at least a portion of the piston portion is slidably positionable at least partially through the at least one outlet external to the reservoir portion, thereby facilitating expulsion of substantially all medicament from the reservoir portion.

4. The medicament applicator of claim 3, wherein the applicator is configured to produce an audible and/or tactile indicator to the user indicating the passage of the at least a portion of the piston portion through the at least one outlet.

5. The medicament applicator of claim 1, wherein the piston portion is configured for substantially sealing an interface between the sliding surfaces of the piston portion and the reservoir portion.

6. The medicament applicator of claim 5, wherein the piston portion includes at least one annular ridge circumferentially disposed around the piston portion for substantially sealing an interface between the sliding surfaces of the piston portion and the reservoir portion.

7. The medicament applicator of claim 5, further comprising a removable cap configured for positioning over the dispensing end portion of the body member, the removable cap including a sealing portion configured to be positioned at least partially through the at least one outlet when the removable cap is positioned over the dispensing end portion of the body member, the sealing portion cooperating with the piston portion to substantially hermetically seal the reservoir portion for keeping air away from any medicament within the reservoir portion and retaining any medicament within the reservoir portion over a predetermined amount of time.

8. The medicament applicator of claim 1, further comprising a removable cap configured for positioning over the dispensing end portion of the body member, the removable cap including a sealing portion configured to be positioned at least partially through the at least one outlet for substantially sealing the at least one outlet when the removable cap is positioned over the dispensing end portion of the body member.

9. The medicament applicator of claim 8, wherein the removable cap includes a recessed portion generally opposite the sealing portion, the recessed portion configured to facilitate compression of the sealing portion for positioning at least partially through at least one outlet.

10. The medicament applicator of claim 8, wherein the removable cap includes an external surface portion configured to facilitate gripping of the removable cap.

11. The medicament applicator of claim 1, wherein the body member includes:
an outer sidewall portion defining at least a portion of the exterior surface of the body member; and
an internal sidewall portion defining at least a portion of the reservoir portion, the internal sidewall portion spaced apart from and generally parallel with the outer sidewall portion.

12. The medicament applicator of claim 1, wherein the at least two forks include end portions having distal camming surfaces oriented so as to urge the at least two forks towards each other when the distal camming surfaces contact one or more surfaces of the second member.

13. The medicament applicator of claim 1, wherein the at least two forks include distal slanted surfaces, wherein the second member includes reciprocally slanted distal surfaces for engaging the respective distal slanted surfaces of the at least two forks, and wherein the distal slanted surfaces are oriented so as to inhibit disengagement of the at least two forks from the second member when a force is applied to the second member in a direction generally opposite the at least one outlet.

14. The medicament applicator of claim 13, wherein the at least two forks further include proximal slanted surfaces, wherein the second member further includes reciprocally slanted proximal surfaces for engaging the respective proximal slanted surfaces of the at least two forks, and wherein said proximal slanted surfaces are oriented so as to inhibit disengagement of the at least two forks from the second member when the first member is slidably moved along with the second member in a direction generally towards the at least one outlet.

15. The medicament applicator of claim 1, wherein the first plunger member includes a protruding portion disposed within the reservoir portion rearward of the piston portion, and wherein the medicament applicator further comprises a stop within the reservoir portion configured for inhibiting sliding movement of the first plunger member in a direction generally opposite the at least one outlet when the protruding portion contacts the stop.

16. The medicament applicator of claim 1, wherein the applicator is configured for holding about one hundred milligrams of medicament within the reservoir portion for delivery to a body cavity.

17. The medicament applicator of claim 1, wherein the applicator is configured for holding about two hundred milligrams of medicament within the reservoir portion for delivery to a body cavity.

18. A medicament applicator comprising:
a body member including a reservoir portion for holding medicament, and at least one outlet in communication with the reservoir portion for dispensing medicament from the reservoir portion;
a plunger including a piston portion slidably disposed within the reservoir portion for urging medicament from the reservoir portion through the at least one outlet as the piston portion slides within the reservoir portion in a direction generally towards the at least one outlet;
the applicator configured such that at least a portion of the piston portion is slidably positionable at least partially through the at least one outlet external to the reservoir portion, thereby facilitating expulsion of substantially all medicament from the reservoir portion.

19. The medicament applicator of claim 18, wherein the applicator is configured to produce an audible and/or tactile indicator to the user indicating the passage of the at least a portion of the piston portion through the at least one outlet.

20. The medicament applicator of claim 19, wherein the piston portion is configured for substantially sealing an interface between the sliding surfaces of the piston portion and the reservoir portion.

21. The medicament applicator of claim 20, wherein the piston portion includes at least one annular ridge circumferentially disposed around the piston portion for substantially sealing the interface between the sliding surfaces of the piston portion and the reservoir portion.

22. The medicament applicator of claim 20, further comprising a removable cap configured for positioning over the dispensing end portion of the body member, the removable cap including a sealing portion configured to be positioned at least partially through the at least one outlet when the removable cap is positioned over the dispensing end portion of the body member, the sealing portion cooperating with the piston portion to substantially hermetically seal the reservoir portion for keeping air away from any medicament within the reservoir portion and retaining any medicament within the reservoir portion over a predetermined amount of time.

23. The medicament applicator of claim 18, further comprising a removable cap configured for positioning over the dispensing end portion of the body member, the removable cap including a sealing portion configured to be positioned at least partially through the at least one outlet for substantially sealing the at least one outlet when the removable cap is positioned over the dispensing end portion of the body member.

24. The medicament applicator of claim 23, wherein the removable cap includes a recessed portion generally opposite the sealing portion, the recessed portion configured to facilitate compression of the sealing portion for positioning at least partially through at least one outlet.

25. The medicament applicator of claim 23, wherein the removable cap includes an external surface portion configured to facilitate gripping of the removable cap.

26. The medicament applicator of claim 18, wherein the body member includes:
an outer sidewall portion defining at least a portion of the exterior surface of the body member; and
an internal sidewall portion defining at least a portion of the reservoir portion, the internal sidewall portion spaced apart from and generally parallel with the outer sidewall portion.

27. The medicament applicator of claim 18, wherein the plunger includes:
a first member including the piston portion and at least two forks, each said fork including at least one interlocking member engagable with at least one interlocking member of the other said fork, whereby engagement of the interlocking members inhibits relative movement of the at least two forks away from each other; and
a second member slidingly associated with the first member, the second member including at least one splitter member for causing disengagement of the engaged interlocking members of the at least two forks.

28. The medicament applicator of claim 27, wherein the at least two forks include end portions having distal camming surfaces oriented so as to urge the at least two forks towards each other when the distal camming surfaces contact one or more surfaces of the second member.

29. The medicament applicator of claim 27, wherein the at least two forks include distal slanted surfaces, wherein the second member includes reciprocally slanted distal surfaces for engaging the respective distal slanted surfaces of the at least two forks, and wherein the distal slanted surfaces are oriented so as to inhibit disengagement of the at least two forks from the second member when a force is applied to the second member in a direction generally opposite the at least one outlet.

30. The medicament applicator of claim 29, wherein the at least two forks further include proximal slanted surfaces, wherein the second member further includes reciprocally slanted proximal surfaces for engaging the respective proximal slanted surfaces of the at least two forks, and wherein said proximal slanted surfaces are oriented so as to inhibit disengagement of the at least two forks from the second member when the first member is slidably moved along with the second member in a direction generally towards the at least one outlet.

31. The medicament applicator of claim 18, wherein the plunger includes a protruding portion disposed within the reservoir portion rearward of the piston portion, and wherein the medicament applicator further comprises a stop within the reservoir portion configured for inhibiting sliding movement of the plunger in a direction generally opposite the at least one outlet when the protruding portion contacts the stop.

32. The medicament applicator of claim 18, wherein the applicator is configured for holding about one hundred milligrams of medicament within the reservoir portion for delivery to a body cavity.

33. The medicament applicator of claim 18, wherein the applicator is configured for holding about two hundred milligrams of medicament within the reservoir portion for delivery to a body cavity.

34. A medicament applicator comprising:
a body member including a reservoir portion for holding medicament, at least one outlet in communication with the reservoir portion for dispensing medicament from the reservoir portion, an outer sidewall portion defining at least a portion of the exterior surface of the body member, and an internal sidewall portion defining at least a portion of the reservoir portion, the internal sidewall portion spaced apart from and generally parallel with the outer sidewall portion;
a plunger including a piston portion slidably disposed within the reservoir portion for urging medicament from the reservoir portion through the at least one outlet as the piston portion slides within the reservoir portion in a direction generally towards the at least one outlet; and
a removable cap configured for positioning over the dispensing end portion of the body member, the removable cap including a sealing portion configured to be positioned at least partially through the at least one outlet for substantially sealing the at least one outlet when the removable cap is positioned over the dispensing end portion of the body member.

35. The medicament applicator of claim 34, wherein the piston portion includes at least one annular ridge circumferentially disposed around the piston portion, the at least one annular ridge cooperating with the sealing portion of the removable cap to substantially hermetically seal the reservoir portion for keeping air away from any medicament within the reservoir portion and retaining any medicament within the reservoir portion over a predetermined amount of time.

36. The medicament applicator of claim 34, wherein the plunger includes:
a first member including the piston portion and at least two forks, each said fork including at least one interlocking member engagable with at least one interlocking member of the other said fork, whereby engagement of the interlocking members inhibits relative movement of the at least two forks away from each other; and
a second member slidingly associated with the first member, the second member including at least one splitter member for causing disengagement of the engaged interlocking members of the at least two forks.

37. The medicament applicator of claim 36, wherein the at least two forks include end portions having distal camming surfaces oriented so as to urge the at least two forks towards each other when the distal camming surfaces contact one or more surfaces of the second member.

38. The medicament applicator of claim 36, wherein the at least two forks include distal slanted surfaces, wherein the second member includes reciprocally slanted distal surfaces for engaging the respective distal slanted surfaces of the at least two forks, and wherein the distal slanted surfaces are oriented so as to inhibit disengagement of the at least two forks from the second member when a force is applied to the second member in a direction generally opposite the at least one outlet.

39. The medicament applicator of claim 34, wherein the plunger includes a protruding portion disposed within the reservoir portion rearward of the piston portion, and wherein the medicament applicator further comprises a stop within the reservoir portion configured for inhibiting sliding movement of the plunger in a direction generally opposite the at least one outlet when the protruding portion contacts the stop.

40. The medicament applicator of claim 34, wherein the applicator is configured for holding about one hundred milligrams of medicament within the reservoir portion for delivery to a body cavity.

41. The medicament applicator of claim 34, wherein the applicator is configured for holding about two hundred milligrams of medicament within the reservoir portion for delivery to a body cavity.
